(19) United States
(12) Patent Application Publication
(10) Pub. No.: US 2014/0017751 A1
(43) Pub. Date: Jan. 16, 2014

(54) METHOD FOR PRODUCING ORGANIC COMPOUNDS VIA FERMENTATION OF BIOMASS AND ZEOLITE CATALYSIS

(71) Applicants: Michael Zavrel, Munich (DE); Oliver Franke, Munich (DE); Oliver Richter, Munich (DE); Michael Kraus, Puchheim (DE)

(72) Inventors: Michael Zavrel, Munich (DE); Oliver Franke, Munich (DE); Oliver Richter, Munich (DE); Michael Kraus, Puchheim (DE)

(73) Assignee: CLARIANT PRODUKTE (DEUTSCHLAND) GMBH, Frankfurt Am Main (DE)

(21) Appl. No.: 13/996,817

(22) PCT Filed: Dec. 23, 2011
(86) PCT No.: PCT/EP2011/073963

§ 371 Date: Oct. 2, 2013

(30) Foreign Application Priority Data
Dec. 23, 2010 (EP) 10196776.8

(51) Int. Cl. C12P 5/02; C12P 5/00

(52) U.S. Cl. CPC C12P 5/026; C12P 5/005
USPC 435/166

(57) ABSTRACT

The invention relates to a method for obtaining organic compounds from biomass, wherein the steps of gas stripping, adsorption from the gas phase, and catalytic reaction are coordinated with each other. The method according to the invention preferably comprises the steps of fermentation, gas stripping, adsorption, desorption, catalytic reaction, condensation, and decantation, which can proceed in parallel. The invention further relates to the coupling of adsorption, desorption, and catalytic reaction by using the same zeolite material for adsorption and catalytic reaction.

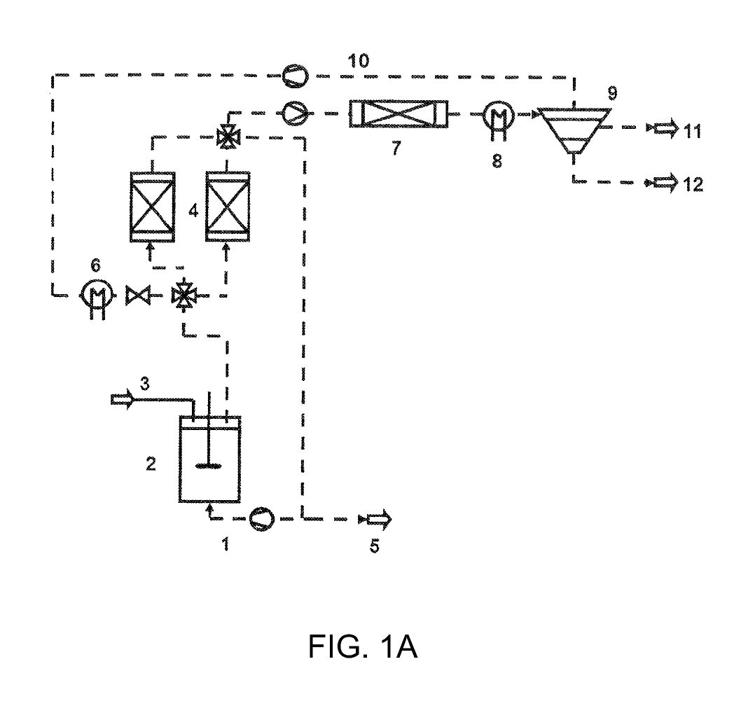

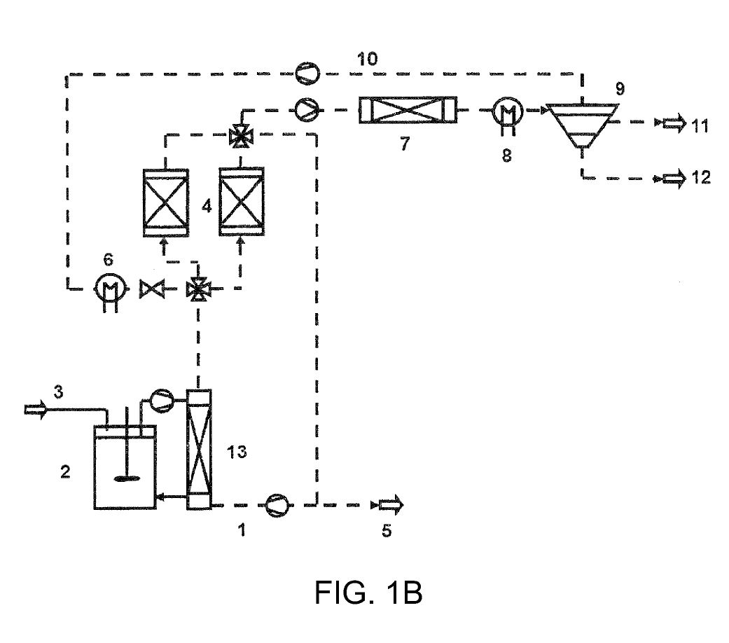

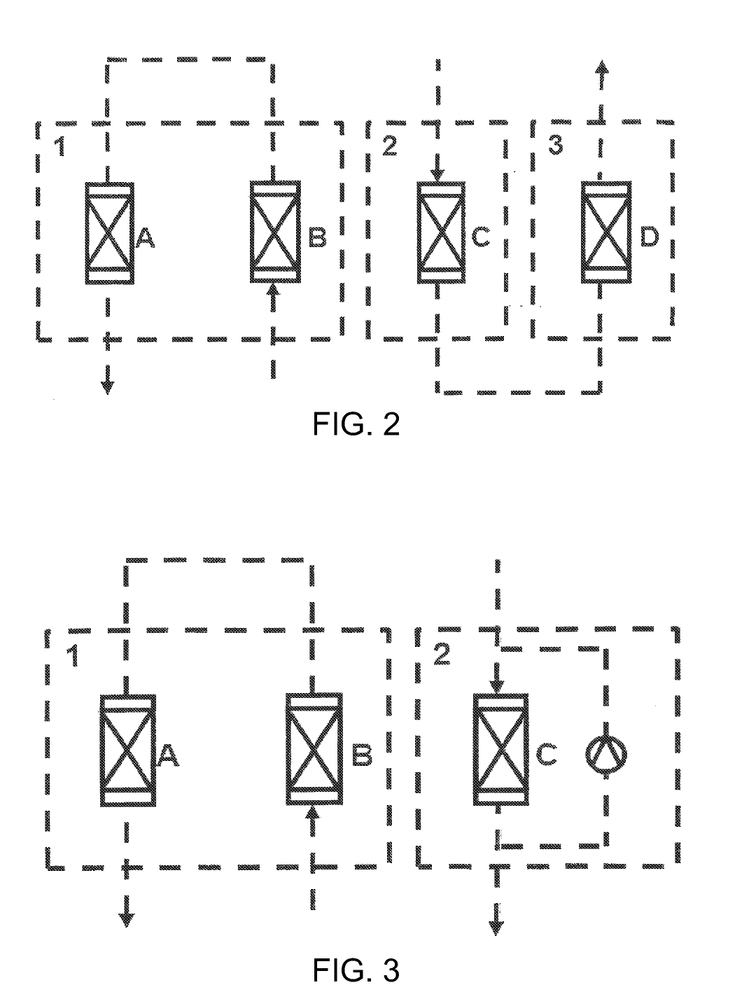

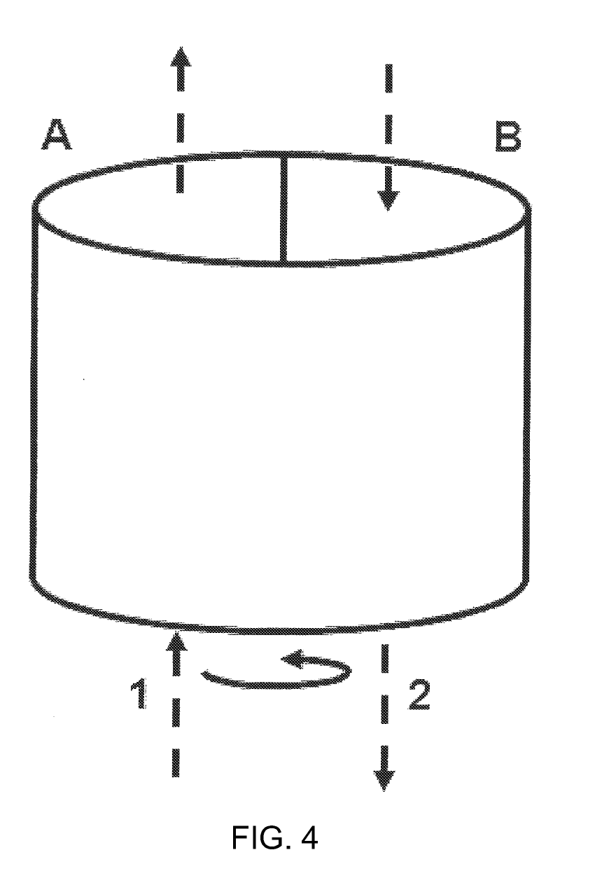

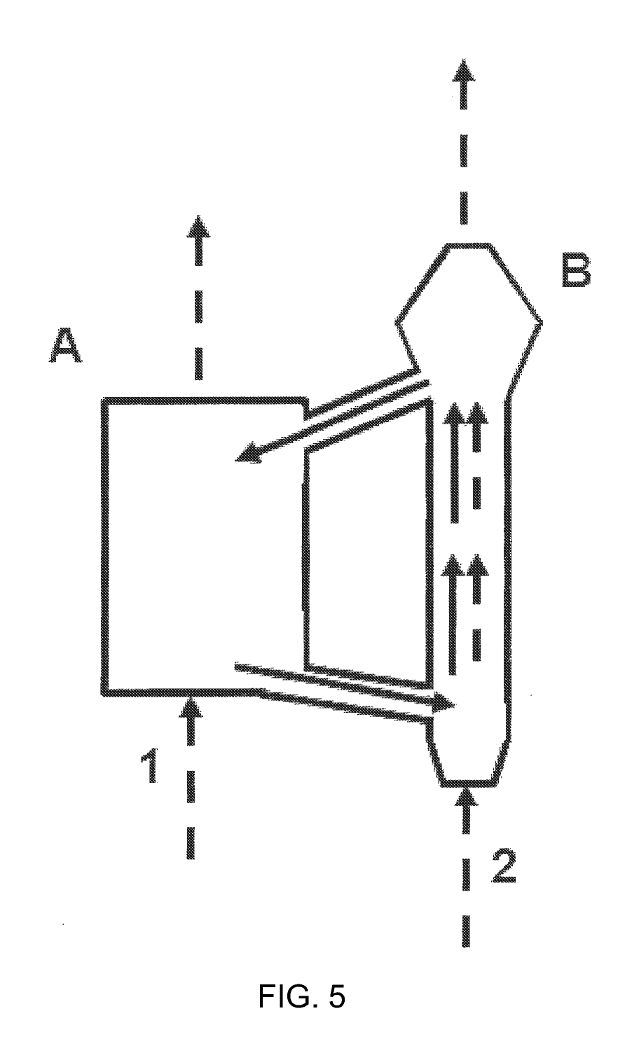

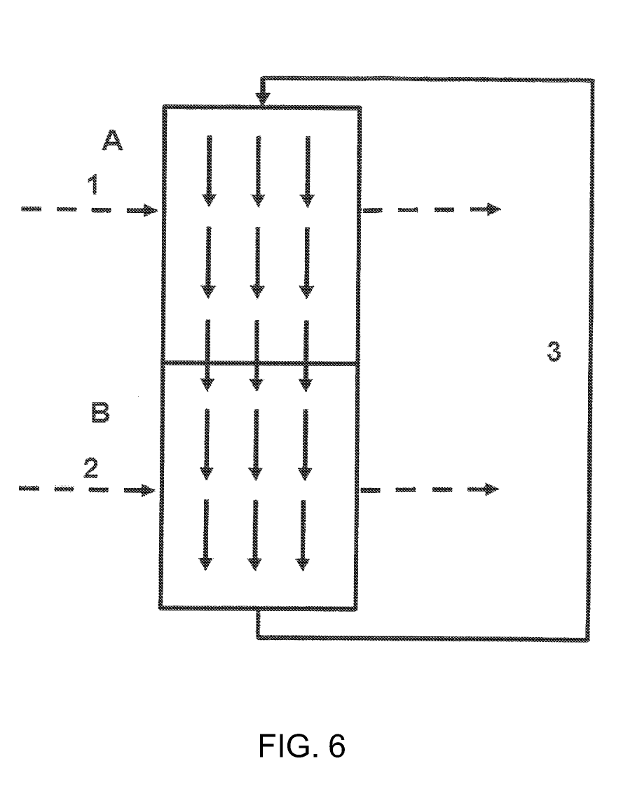

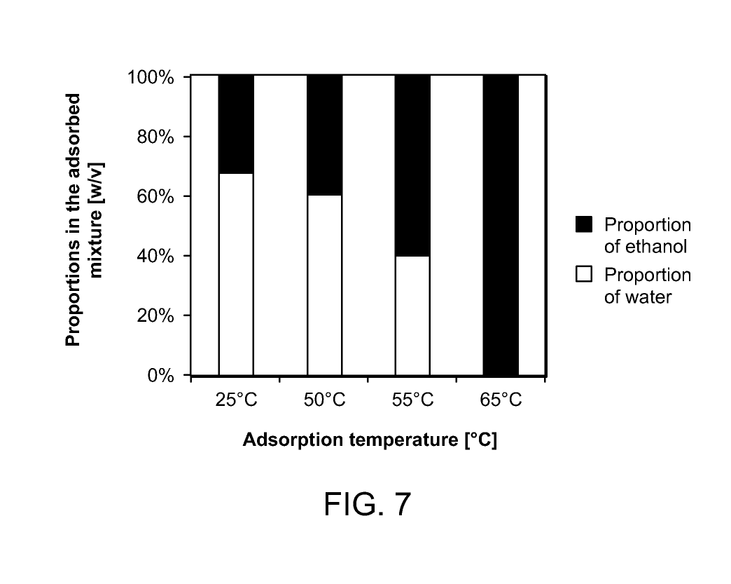

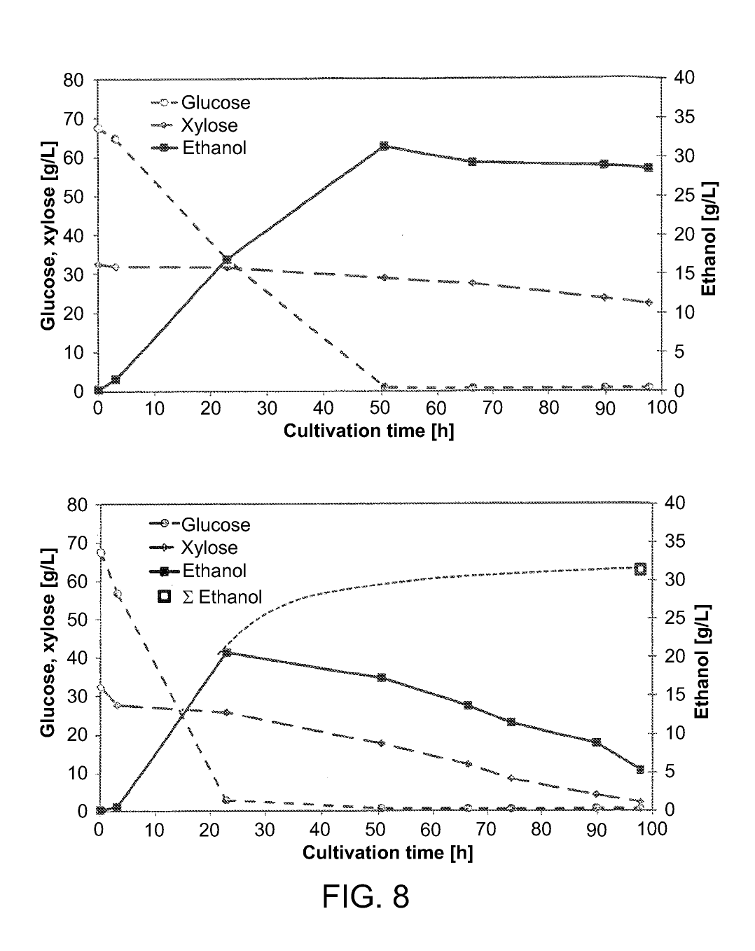

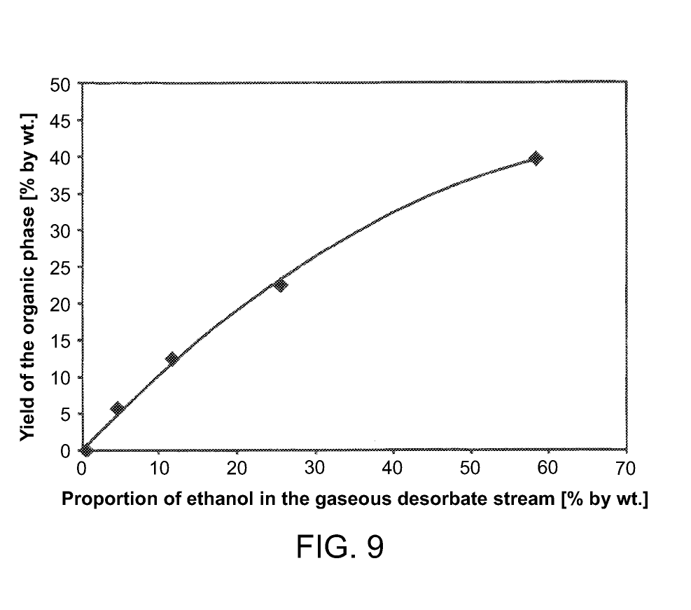

### FIELD OF THE INVENTION

The invention relates to a method for producing organic compounds from biomass.

### PRIOR ART

Processes enabling the production of organic compounds from fermentation-derived alcohol are known from literature. They basically comprise the steps of sugar fermentation, distillation of the fermentation medium, catalytic conversion of the thermally separated alcohol to organic compounds, and separation of the organic compounds from the process water (see, e.g., U.S. Pat. No. 3,936,353; CA 2,360,981).

In deviation from this, it is possible, according to the patent specification U.S. Pat. No. 4,690,903, to obtain the fermentation-derived alcohol from the fermentation broth by sorption to an adsorbent which occurs directly in the fermentation broth. In case the adsorbent is a zeolite, it is optionally possible to convey the loaded zeolite into a reaction zone in which the sorbed alcohol is catalytically converted to organic compounds by means of the zeolite.

Dehydration reactions are particularly suitable for converting alcohols to organic compounds having a lower oxygen/carbon ratio. MFI-type zeolites in the hydrogen form (H-ZSM-5, SiO₂/Al₂O₃>10) are described in literature as catalysts for this dehydration of alcohols (mostly ethanol) (see, e.g., U.S. Pat. No. 3,936,353; U.S. Pat. No. 4,690,903; U.S. Pat. No. 4,621,164; Oudejans et al., App. Catalysis Vol. 3, 1982, p. 109; Aguayo et al., J. Chem. Technol. Biotechnol. Vol. 77, 2002, p. 211). Furthermore, modifications of the zeolite H-ZSM-5 by, e.g., impregnation with metals/metal oxides or phosphoric acid are also known which allow the selectivity of the conversion to ethene (U.S. Pat. No. 4,698,452) or also the selectivity of the conversion to aromatics (WO 2007/137566 A1) to be influenced. Besides H-ZSM-5 zeolites, other types of zeolites (U.S. Pat. No. 4,621,164; Oudejans et al., App. Catalysis Vol. 3, 1982, p. 109), mesoporous molecular sieves (Varisli et al.; Chem. Eng. Sci. Vol. 65, 2010, p. 153) and hydroxyapatite (Tsuchida et al., Ind. Eng. Chem. Res. Vol. 47; 2008, p. 1443) have also been studied as further catalysts for ethanol dehydration.

According to the prior art, dehydration takes place in a packed bed reactor at temperatures between 150° C. and 500° C., absolute pressures of 1 bar to 100 bar, and liquid hourly space velocities (LHSV=reactant liquid flow rate/catalyst volume) in the range of from 0.5 11⁻¹ to 50 h⁻¹ (see, e.g., U.S. Pat. No. 4,621,164; Oudejans et al., App. Catalysis Vol. 3, 1982, p. 109).

It is possible by adding water to the ethanol input stream to increase the proportion of aromatics in the product stream and to reduce catalyst deactivation due to coking (Oudejans et al., App. Catalysis Vol. 3, 1982, p. 109). The yield of liquid organic compounds may likewise be influenced by varying the proportion of water (U.S. Pat. No. 4,621,164). A low proportion of water gives rise to a high proportion of organic compounds and vice versa.

WO 2008/066581 A1 describes a method for producing at least one butene, wherein butanol and water are reacted. Here, the reagent may originate from a fermentation broth, with it being possible in one embodiment to use gas stripping to this end. This gas stream is either directly used for reaction or is previously subjected to distillation.

All of the state-of-the-art methods for producing organic compounds from sugars are disadvantageous in that volatile fermentation by-products (e.g. furans) and volatile additives normally used during fermentation (e.g. ammonia as pH adjusting agent) cannot be selectively separated. During the subsequent catalytic reaction, these result in deactivation of the (zeolite) catalyst and thus in a reduction of catalyst activity and selectivity (see, e.g., Hutchings, Studies in Surface Science and Catalysis Vol. 61, 1991, p. 405).

It is likewise disadvantageous that, according to the prior art, the fermentation required for producing the alcohol cannot be directly coupled to the catalytic reaction. With higher concentrations of the intermediate alcohol, fermentation is, however, normally inhibited, thereby limiting the organic compound yield and productivity (space-time yield). Dominguez et al. (Biotech. Bioeng., 2000, Vol. 67, pp. 336-343), for example, show that the conversion of C5 sugars to ethanol by the yeast Pichia stipitis is inhibited in only 2% (w/v) of ethanol. Likewise, when using Clostridia for acetone, butanol and ethanol fermentation, an inhibiting and increasingly toxic influence of the formed products can be observed, such that butanol concentrations of 1.5% (w/v) are not generally exceeded (Häggström L., Biotech. Advs., 1985, Vol. 3, pp. 13-28).

An additional disadvantage of using zeolites for sorption of the alcohol in the fermentation medium is that the sorption capacity of the zeolite decreases with increasing life span on account of fouling processes. Also, the separation of the zeolite from further solids contained in the fermentation medium (e.g. cells, by-products of metabolism, components of nutrient media) is technically complex. Another disadvantage of the thermal separation processes for separating the alcohol from the fermentation medium described in the prior art is that with single distillation the composition of the distillate stream is limited by the initial concentration and the thermodynamic equilibrium of the substances. The composition of the distillate stream can be varied by using multiple distillation, or rectification. However, it is particularly disadvantageous here that higher energy input becomes necessary as a result of the multiple distillate condensation that is due to the very nature of the process.

### SUMMARY OF THE INVENTION

In view hereof, it is the object of the present invention to develop an economical method for producing organic compounds from biomass which overcomes the disadvantages of the prior art and allows a high yield of organic compounds to be achieved whilst keeping the complexity of the required equipment as low as possible.

This problem has surprisingly been solved by the combination of fermentation with product separation via gas stripping, adsorption, desorption and catalytic reaction, which makes it possible to convert biomass to organic compounds and in which all method steps may proceed in parallel.

A method for producing organic compounds is thus provided according to the invention which comprises the following steps:
a. fermentative conversion of biomass to volatile organic compounds in a bioreactor;
b. removal of the volatile organic compounds by gas stripping using a carrier gas;
c. adsorption of the volatile organic compounds from the gas stream;
d. desorption of the adsorbed volatile organic compounds from the adsorbent;
e. catalytic reaction of the volatile organic compounds.

In method step d, the proportion of volatile organic compounds in the desorbate stream preferably lies between 10% (w/w) and 90% (w/w), especially preferably between 30% (w/w) and 70% (w/w), and even more preferably between 35% (w/w) and 60% (w/w).

The products of the catalytic reaction can then be processed, for example, by condensation of the product stream and phase separation, preferably via decantation.

### DETAILED DESCRIPTION OF THE INVENTION

Within the scope of this invention, a method for producing organic compounds is provided, comprising the following method steps:
a. fermentative conversion of biomass to volatile organic compounds in a bioreactor;
b. removal of the volatile organic compounds by gas stripping using a carrier gas;
c. adsorption of the volatile organic compounds from the gas stream;
d. desorption of the adsorbed volatile organic compounds from the adsorbent;
e. catalytic reaction of the volatile organic compounds.

The individual method steps are described in more detail below:

a. Fermentation

A solution comprising biomass is provided for fermentation. Biomass is thereby understood to mean biologic material comprising one or more of the following components: cellulose, hemicellulose, lignin, pectin, starch, sucrose, chitin, proteins and other biopolymers, as well as fats and oils. Furthermore, this term also includes biologic materials containing sugars, particularly C5 and C6 sugars, amino acids, fatty acids and other biologic monomers, or from which these monomers can be obtained, preferably by hydrolysis. At the beginning of fermentation, the solution preferably contains less than 200 g/L sugar, especially preferably less than 100 g/L sugar. In a preferred embodiment, the solution contains sugars derived from lignocellulosic biomass and especially preferably from previous enzymatic hydrolysis. An equally preferred procedure is the combination of fermentation with enzymatic hydrolysis such that hydrolysis and fermentation take place simultaneously. This means that, if fermentation takes place at the same time as the subsequent steps, as in the preferred embodiment described further below, these embodiments can also be combined, meaning that both hydrolysis and fermentation proceed at the same time as the subsequent steps.

In another preferred embodiment, the fermentation solution contains one or more low-molecular carbon sources, as well as optionally one or more low-molecular nitrogen sources. Preferred low-molecular carbon sources are monosaccharides such as glucose, fructose, galactose, xylose, arabinose, mannose, disaccharides such as sucrose, lactose, maltose, cellobiose, saccharic acids such as galacturonic acid, gluconic acid, polyols such as glycerin, sorbitol, as well as oils, fats and fatty acids. Preferred nitrogen sources are ammonia, ammonium salts, nitrate salts, amino acids, urea, and hydrolized proteins. Low-molecular is understood to mean that the molecular weight is preferably less than 2500 and especially preferably less than 1000.

Ammonia is to be especially preferred as a nitrogen source since it serves at the same time as a pH adjusting agent, i.e. it can be added if the pH value is too low before fermentation. Furthermore, it is also possible in a particular embodiment to add ammonia during fermentation if the pH value drops as a result of the metabolic activity of the fermented microorganisms. This allows the pH to be adjusted or regulated throughout the entire duration of the fermentation. Further additives such as other pH adjusting agents and anti-foaming agents can be added to the fermentation solution, in addition to microorganisms and enzymes. Yeasts, fungi and/or bacteria are suitable microorganisms. Microorganisms which produce alcohols, ketones, aldehydes and/or organic acids are preferred. Slightly volatile organic compounds such as ethanol and/or acetone and/or butanols are particularly preferred products. Volatile compound is thereby understood to mean a compound having a vapour pressure greater than 1.0 hPa, preferably greater than 5.0 hPa, at 20° C. This includes compounds which at 20° C. have a vapour pressure equal to or greater than that of 1-butanol, such as, for example 2-butanol, tert-butanol, ethanol, 1-propanol, isopropanol and acetone. This means that, in a preferred embodiment, the present invention comprises a method that is furthermore characterized in that the volatile organic compounds are alcohols and/or ketones and/or aldehydes and/or organic acids, preferably ethanol and/or butanol and/or acetone. Unless specified otherwise, butanol includes all butanols, with 1-butanol being especially preferred, however.

The fermentation typically takes place at temperatures between 10 and 70° C., preferably between 20 and 60° C., especially preferably between 30 and 50° C. The fermentation is preferably run in the batch operation mode. In another preferred embodiment, nutrition medium is continuously fed in during fermentation (fed-batch operation). It is furthermore preferred for the fermentation to be run in continuous mode. The repeated-batch and repeated-fed-batch modes, as well as two-step procedures and cascades, are also preferred.

The fermentation can be carried out by isolated enzymes that are added to the fermentation solution. However, it is preferred for the fermentation to be carried out by means of at least one microorganism. This at least one microorganism is preferably selected from mesophilic and thermophilic organisms. The mesophilic as well as thermophilic organisms may in turn be selected from the group consisting of bacteria, archaea and eukaryotes, with the eukaroytes being particularly preferably fungi and even more preferably yeasts. The yeasts used are most preferably mesophilic yeasts such as, for example, Saccharomyces cerevisiae, Pichia stipitis, Pichia segobiensis, Candida shehatae, Candida tropicalis, Candida boidinii, Candida tenuis, Pachysolen tannophilus, Hansenula polymorpha, Candida famata, Candida parapsilosis, Candida rugosa, Candida sonorensis, Issatchenkia terricola, Kloeckera apis, Pichia barkeri, Pichia cactophila, Pichia deserticola, Pichia norvegensis, Pichia membranaefaciens, Pichia Mexicana and Torulaspora delbrueckii. Examples of mesophilic bacteria include Clostridium acetobutylicum, Clostridium beijerincki, Clostridium saccharobutylicum, Clostridicum saccharoperbutylacetonicum, Escherichia coli, Zymomonas mobilis. In an alternative, particularly preferred embodiment, use is made of thermophilic organisms. Examples of thermophilic yeasts include Candida bovina, Candida picachoensis, Candida emberorum, Candida pintolopesii, Candida thermophila, Kluyveromyces marxianus, Kluyveromyces fragilis, Kazachstania telluris, Issatchenkia orientalis and Lachancea thermolerans. Thermophilic bacteria include, inter alia, Clostridium thermocellum, Clostridium thermohydrosulphuricum, Clostridium thermosaccharolyticium, Thermoanaerobium brockii, Thermobacteroides acetoethylicus, Thermoanaerobacter ethanolicus, Clostridium thermoaceticum, Clostridium thermoautotrophicum, Acetogenium kivui, Desulfotomaculum nigrificans, and Desulfovibrio thermophilus, Thermoanaerobacter tengcongensis, Bacillus stearothermophilus and Thermoanaerobacter mathranii. In an alternative, also preferred embodiment, use is made of microorganisms that have been modified by genetic methods.

b. Gas Stripping

According to the present invention, the volatile components, particularly the volatile organic products, are transferred to the gas phase by stripping with a carrier gas. During gas stripping, also referred to as stripping, volatile compounds are removed from the liquid phase by passing gas therethrough, and are transferred to the gaseous phase. In a preferred embodiment, this transfer may take place continuously. Continuous removal of the volatile components thereby refers to the removal of the volatile components by gas stripping, in parallel to the production thereof by fermentation. Inert gases such as, for example, carbon dioxide, helium, hydrogen, nitrogen or air, as well as mixtures of these gases, may come into consideration as carrier gas. Gases which are very poorly reactive, i.e. capable of participating in only a few chemical reactions, are thereby considered to be inert. Particular preference is given to carbon dioxide and mixtures of carbon dioxide and air, which allow microaerobic conditions to be set as needed. One advantage of the method according to the invention consists in the fact that the fermentation exhaust gases formed during fermentation can be directly used as carrier gas. It is also preferred in a particular embodiment that the fermentation exhaust gases are employed as carrier gas.

In accordance with the method according to the invention, fermentation and gas stripping take place in a reactor that is preferably selected from the group consisting of a stirred-tank reactor, a loop reactor, an airlift reactor or a bubble column reactor. Dispersal of the gas bubbles, which may be achieved, for example, by means of a sparger and/or an appropriate stirrer, is particularly preferred. In addition, gas stripping is possible via an external gas stripping column connected to the bioreactor which is optionally fed continuously with the fermentation solution and the output of which can be returned into the bioreactor. It is especially preferred for such an external gas stripping column to be operated in the counter-current mode and/or in combination with packing, preferably with Raschig rings, to increase the mass transfer rate.

The specific gassing rate (gas volume flow) preferably lies between 0.1 and 10 vvm, especially preferably between 0.5 and 5 vvm (vvm means gas volume per bioreactor volume per minute). The gas stripping is preferably carried out at a pressure between 0.05 and 10 bar, especially preferably between 0.5 and 1.3 bar. The gas stripping is even more preferably carried out at sub-atmospheric pressure (or negative overpressure), i.e. at a pressure lower than the reference pressure of the surroundings which is typically about 1 bar. The gas stripping preferably takes place at fermentation temperature. In an alternative embodiment, which is also preferred, the gas stripping occurs such that the fermentation solution is additionally heated. This may be achieved by using a set-up in which a portion of the fermentation solution is directed into an external column in which the temperature is increased and in which the gas stripping takes place, which makes the gas stripping more efficient than at fermentation temperature.

Another advantage of the method according to the invention consists in the fact that the heat of vaporization which is carried away due to the transition of the volatile compounds from a liquid into a gas phase contributes to cooling of the bioreactor, thus reducing the cooling capacity required to keep the temperature in the bioreactor constant. In a particularly preferred embodiment of the method according to the invention, absolutely no cooling is required since the sum of the dissipated heat of vaporization and the heat lost to the surroundings is greater than the biologically produced heat.

c. Adsorption

According to the method of the invention, the gas stream exiting the bioreactor is directed through one or more columns filled with one or more adsorbents. Suitable adsorbents are zeolites, silica, bentonites, silicalites, clays, hydrotalcites, alumino-silicates, oxide powders, mica, glass, aluminates, clinoptolite, gismondine, quartzes, activated carbons, bone char, montmorillonites, polystyrenes, polyurethanes, polyacrylamides, polymethacrylates and polyvinyl pyridines, or mixtures thereof. In a preferred embodiment, zeolites are used as adsorbents. Beta or MFI type zeolites are particularly preferred. The zeolite preferably has a SiO₂/Al₂O₃ ratio of 5 to 1000, and particularly preferably a SiO₂/Al₂O₃ ratio of 100 to 900. The synthetic zeolites according to U.S. Pat. No. 7,244,409 are especially preferred.

The mass ratio of adsorbent to adsorbed ethanol preferably lies between 1 and 1000, especially preferably between 2 and 20. The temperature during the adsorption of ethanol preferably lies between 10 and 100° C., especially preferably between 20 and 70° C. The pressure preferably lies between 0.5 and 10 bar, especially preferably between 1 and 2 bar.

The adsorbing material may be contained in one or more columns. Preferably several columns are used, especially preferably 2 or more, and even more preferably 2 to 6 columns. These columns may be connected in series or in parallel. The advantage of parallel connection is that it enables near-continuous operation in that two or more columns alternate between the adsorption and the desorption described in more detail in point d, meaning that the adsorption and desorption may be carried out simultaneously in different columns. The columns are preferably provided in a revolver arrangement. In a particularly preferred embodiment, 2 to 6 columns are connected such that the column(s) in which the adsorption occurs is/are connected in parallel to the column(s) in which the desorption occurs. Where the adsorption occurs in more than one column, these columns may be connected in series or in parallel. Thus, when using, for example, 6 columns in the “revolver” configuration, the adsorption may occur in columns 1 to 3 while column 4 is being heated for desorption, and desorption may occur in column 5 while allowing column 6 to cool. The adsorption column is changed when the adsorbent loading reaches a predetermined value, at the latest though when the full loading has been attained and when the volatile organic compounds break through at the end of the column, i.e. can no longer be fully adsorbed.

The gas stream typically contains more water than volatile organic compounds, and therefore the adsorbents first of all saturate with water. Loading with the volatile organic compounds then increases continuously over a second period of time until saturation is reached here as well. During this second period of time, the ratio of volatile organic compounds to water rises continuously. Having regard to the subsequent catalytic reaction, a particularly preferred embodiment of the method consists in setting this ratio between volatile organic compounds and water in such a manner—by selecting a suitable cycle time and/or a suitable amount of adsorbent—as to allow for a particularly suitable mixing ratio, i.e. a proportion of volatile organic compounds that is particularly suitable or optimal for the catalytic reaction. The cycle times and/or amounts of adsorbent that are particularly beneficial or optimal for this can be determined by preliminary experiments. Particularly suitable proportions of volatile organic compounds lie between 10% (w/w) and 90% (w/w), especially preferably between 30% (w/w) and 70% (w/w), and even more preferably between 35% (w/w) and 60% (w/w). The residual proportions are made up of water and/or carrier gas.

The adsorption material used is preferably capable of selective adsorption. Selective adsorption to an adsorption material is thereby understood to mean that the adsorption material is capable of adsorbing a higher mass fraction of the desired compound than of the undesired compound from a gas stream. Desired compounds within the meaning of this invention are the volatile organic compounds. Undesired compounds within the meaning of this invention are, for example, catalyst poisons such as ammonia, as will be specified in the next section. This means that, if the gas stream consists of equal mass fractions of volatile organic compounds and undesired compound, more is absorbed of the volatile organic compounds than of the undesired compound. A preferred ratio of volatile organic compound to undesired compound is at least 5:1, especially preferably at least 20:1.

In a preferred embodiment, the adsorbing material is selected so that only negligible or non-measurable amounts of undesired compounds, such as, e.g., catalyst poisons, are adsorbed for the subsequent catalytic reaction Ammonia, furans, furfural, as well as derivatives thereof such as hydroxymethylfurfural (HMF), are typical undesired compounds that may act as catalyst poisons, alone or in combination. In a particularly preferred embodiment, adsorption of ammonia is avoided either completely or to a very large extent, provided that an adsorbing material having only a few acid sites is employed. For example, zeolites having a SiO₂/Al₂O₃ ratio of at least 100 are suitable for this. These zeolites are therefore particularly preferred as adsorbing material for this embodiment. If both the adsorbent and the catalyst are zeolites, it is preferred in one embodiment for the adsorbent to have a SiO₂/Al₂O₃ ratio greater than that of the catalyst.

Examples 4 and 5 together show that zeolite is suited for selective adsorption of ethanol and that adsorption of the undesired compound ammonia is negligible.

The gas stream depleted of volatile organic compounds exits the adsorber. Given that selective adsorption is made possible as described above, the previously described undesired compounds, upon this exit, are depleted or removed from the product stream which is then further processed, as will be described in d. and e. below. The step d thus makes it possible—unlike, e.g., the optional distillation described in WO 2008/066581 A1 —to efficiently deplete or remove undesired compounds. Following its exit from the adsorption column, the gas stream can be recirculated into the bioreactor and is then available once again for gas stripping. The adsorption may be carried out in the fluidised bed mode. Radial adsorbers or rotary adsorbers may equally be employed. Since the recirculated gas stream in this embodiment is depleted of organic compounds, the concentration of volatile organic compounds can be kept low in the fermentation medium, despite the gas recirculation.

The combination according to the invention of in situ gas stripping and adsorption to zeolite allows the concentration of volatile organic compounds in the fermentation solution to be kept below a specific value throughout the entire duration of the fermentation process. This is particularly preferred if the volatile organic compounds exert an inhibiting or toxic effect on the microorganisms, as is the case, for example, for ethanol, butanol or acetone. The adsorption is preferably carried out at least throughout the entire duration of the production of the volatile organic compounds, i.e. for as long as these volatile organic compounds are being produced. A low concentration of volatile organic compounds in the fermentation medium means, for example, a total amount of volatile organic compounds in the fermentation medium of less than 10% (w/v), preferably less than 5% (w/v) of volatile organic compounds in the fermentation medium, particularly preferably less than 3.5% (w/v) of volatile organic compounds in the fermentation medium, and most preferably less than 2% (w/v) of volatile organic compounds in the fermentation medium. As regards the individual components, the amount of ethanol present in the fermentation medium is preferably less than 10% (w/v) and more preferably less than 5% (w/v), and the amount of butanol present in the fermentation medium is preferably less than 3% (w/v), more preferably less than 2%, and even more preferably less than 1.5% (w/v), wherein, for the purposes of that stated in this sentence, butanol includes the sum of all butanols, i.e. 1-butanol, 2-butanol and tert-butanol.

d. Desorption

The method according to the invention enables desorption of volatile organic compounds from the adsorbent. In step d. of the method according to the invention, the proportion of volatile organic compounds in the desorbate stream thereby preferably lies between 10% (w/w) and 90% (w/w), especially preferably between 30% (w/w) and 70% (w/w), and even more preferably between 35% (w/w) and 60% (w/w).

Desorption may occur by increasing temperature and/or reducing pressure within the column. Temperatures between 25 and 300° C. and absolute pressures between 0 and 10 bar are preferred. Temperatures between 80 and 300° C., as well as absolute pressures between 0.1 and 3 bar, are especially preferred.

In a preferred embodiment of the method according to the invention, a carrier gas is used for carrying the desorbed volatile organic compounds out of the column. The same inert carrier gas is especially preferably used here which is also employed for gas stripping. The “same carrier gas” means that a gas of the same type is employed. To illustrate this: If, for example, the carrier gas in step b. is a gas A (which may be carbon dioxide), the gas in the embodiment of the “same” carrier gas will also be the gas A (which may be carbon dioxide) in step d. What is important, however, is that the gas stream used in step d. is preferably not the same as that used in step b. The reason is that the gas stream used in step b. typically contains undesired compounds in the subsequent step c., i.e. upon exiting the adsorber, as is described above. As a result, the gas stream which is used in step d. for desorption and is then subjected to step e. described below can be depleted of undesired compounds. In another preferred embodiment of the method according to the invention, the temperature and absolute pressure of the carrier gas are set in the column so as to correspond to the temperatures and absolute pressures described above. Upstream heat exchangers and/or chokes or compressors are suited for this purpose.

Desorption may be carried out in the fluidised bed mode. Radial adsorbers or rotary adsorbers may equally be employed.

e. Catalytic Reaction

In accordance with the present invention, the desorbate stream described in section d is transferred into one or more reactors filled with catalyst, with it being optionally possible to bring the input stream to the reaction temperature and reaction pressure by means of upstream heat exchangers and chokes or compressors. Depending on the selected reaction conditions, individual organic compounds or mixtures thereof, which can be allocated, inter alia, to the groups of olefins, aliphates, aromatics, oxygenates, are produced in the reactor.

Fluidised bed reactors, radial flow reactors, entrained flow reactors, moving bed reactors, loop reactors or packed bed reactors can be preferably employed as reactors. These reactors will be briefly described within the framework of the preferred embodiments of this invention. Likewise, it is possible for several reactors of the same or of different structural designs to be combined.

Suitable catalysts are acid substances of the Bronsted and/or Lewis type such as, for example, zeolites, silica-aluminas, aluminas, mesoporous molecular sieves, hydroxyapatites, bentonites, sulfated zirconia, and silicon alumophosphates. In a preferred embodiment, zeolites are used as catalysts. MFI-type zeolites in the hydrogen form (H-ZSM-5) are preferred zeolites. The zeolite preferably has a SiO₂/Al₂O₃ ratio equal to or greater than 5, such as, for example, of from 5 to 1000, and especially preferably has a SiO₂/Al₂O₃ ratio of from 20 to 200. If both the adsorbent and the catalyst are zeolites, the catalyst zeolite preferably has a SiO₂/Al₂O₃ ratio lower than the adsorbent zeolite. Particularly in this embodiment, but not limited thereto, the catalyst zeolite has a SiO₂/Al₂O₃ ratio of values smaller than 100.

Reaction conditions that are preferred for the catalytic reaction are a temperature of 150 to 500° C., absolute pressures of 0.5 to 100 bar, and a gas hourly space velocity (GHSV=Reactant gas flow rate/catalyst volume) of 100 to 20000 h⁻¹. In a particularly preferred embodiment, the temperature lies in a range of from 250 to 350° C., the absolute pressure in a range of from 1 to 5 bar, and the GHSV in a range of from 2000 to 8000 h⁻¹.

One advantage of the method according to the invention over the prior art lies in the combination of the adsorption/desorption described in sections c/d with the catalytic reaction described herein. Due to the targeted selection of the adsorption and desorption conditions, it has become possible for the first time to adjust the proportion of water, as well as the proportion of volatile organic compounds, in the desorbate stream and thus in the input stream of the catalytic reaction. It is possible by appropriately selecting the proportion of volatile organic compounds to significantly influence the yield of liquid organic compounds and by appropriately selecting the proportion of water to significantly influence the catalyst's deactivation characteristics. The combination of the adsorption/desorption described in sections c/d with the catalytic reaction described herein also allows undesired compounds to be removed from the gas stream. This avoids exposing the catalyst to catalyst poisons to such an extent as would the case, for example, in the method according to WO 2008/066581 A1 which does not include an adsorption process.

The catalytic reaction preferably takes place at a temperature of 150 to 500° C., preferably between 250 and 350° C., an absolute pressure of 0.5 to 100 bar, preferably between 1 and 5 bar, and a GHSV of 100 to 20000 h⁻¹, preferably between 2000 and 8000 h⁻¹.

In a preferred embodiment, the proportion of volatile organic compounds in the input stream ranges from 10 to 90% (w/w), in a particularly preferred embodiment from 30 to 70% (w/w), and in an even more preferred embodiment from 35 to 60% (w/w). The respective residual proportions adding to 100% (w/w) are composed of the proportion of water and/or the carrier gas.

f. Condensation

In a preferred embodiment, the method according to the invention can moreover be characterized in that, following method steps a to e described above, condensation of the product stream takes place, which may optionally be achieved by temperature reduction and/or pressure increase. Temperature reduction to a temperature level below ambient temperature, and especially preferably below 10° C., is preferred thereby. Heat exchangers operated in the parallel flow, counter flow or cross flow mode can be employed for this cooling. In accordance with a preferred embodiment of the method according to the invention, condensation takes place gradually, such that several fractions having different compositions are obtained.

The present invention also comprises a method that is further characterized in that the carrier gas(es) can be recirculated following adsorption and/or catalytic reaction. Here, it is preferred for the fermentation exhaust gases to be employed as carrier gas. The non-condensable gas stream fractions are preferably subjected to further catalytic reaction, preferably by being recirculated into the catalytic reaction column.

According to another preferred embodiment of the method according to the invention, these non-condensable fractions are used as reactants for one or more other chemical reactions such as polymerisation reactions. Polymerisation of ethylene to polyethylene or of propene to polypropylene is particularly preferred. According to another preferred embodiment, the non-condensable fractions are used for recovery of heat energy in that they are incinerated. In all of these embodiments, it is also possible to carry out a further adsorption process followed by a desorption process, to enrich the components. A zeolitic material is preferably employed thereby as the adsorbent. The same material should particularly preferably be used here as for the method steps described in c and/or e.

In accordance with the method of the invention, the condensate which forms is collected. In a preferred embodiment, the condensate which forms is kept cool in order to avoid loss due to evaporation.

g. Phase Separation

In another preferred embodiment, the method described in f can furthermore be characterized in that phase separation occurs following condensation. Owing to the miscibility gap between the organic compounds and water, two phases, an organic and an aqueous phase, are preferably formed following condensation. According to the method of the invention, the phases are separated from one another. This can simply be achieved by decantation or centrifugation or any other liquid-liquid separation method known to those skilled in the art. In a particularly preferred embodiment, the organic compounds, as the lighter phase, i.e. lighter than the aqueous phase, are separated during decantation. A particular advantage of the method according to the invention lies in the fact that a large amount of water can thus be separated from the product without high energy input.

The aqueous phase can be returned to other method steps in the form of process water. According to a preferred embodiment, the aqueous phase is rid of any volatile hydrocarbons that may still be dissolved therein by gas stripping. According to a particularly preferred embodiment of the method, these volatile hydrocarbons are recirculated so as to undergo either the adsorption of section c or the catalytic reaction of section e, wherein the carrier gas stream used is either the same carrier gas stream as used for the bioreactor gas stripping process or the same carrier gas stream as used for the catalytic reaction.

The organic phase can be obtained either directly or as a product following further processing. Another preferred type of processing is the separation of the organic mixture into several fractions and/or components which may each be used in different ways.

Use of the product, or of fractions thereof, as fuel or as additive to fuels is particularly advantageous. Fuels may be petrols, diesel fuels, aviation fuels, or similar fuels. Moreover, the product may be used as a fuel such as, for example, fuel oil. An alternative use according to the invention is further use for subsequent chemical reactions, particularly preferably for the production of polymers.

Parallel Set-Up

The method according to the invention in general, as well as the embodiments thereof which additionally include the steps f and g described above, may furthermore be characterized in that the method steps a to e proceed in parallel. Particularly preferred, though not limiting embodiments in this regard are specified hereinbelow:

### Particularly Preferred Embodiments

Illustration 1a shows a possible embodiment of the method according to the invention. An inert carrier gas stream (1) is blown into the bioreactor (2) for gas stripping. Biomass is fermented in the bioreactor to give volatile organic compounds, with auxiliaries (3) such as pH adjusting agents being added. The gas exiting the bioreactor, containing volatile organic compounds and other volatile components, is passed through an adsorption column (4) in which the volatile organic compounds are selectively adsorbed. The depleted gas stream is then recirculated into the bioreactor. To ensure near-continuous operation, two or more columns are connected in parallel and/or in series. A portion of the carrier gas stream is discharged as a result of the fermentation exhaust gases generated during fermentation (5). The temperature and/or pressure within the column (4) is changed for desorption of the adsorbed organic compounds. The carrier gas stream (10) necessary for carrying out the desorbed volatile organic compounds is appropriately adjusted via a heat exchanger (6) and/or chokes.

The gas exiting the column upon desorption is then catalytically reacted in one or more reactors (7). The organic products thus formed are condensed via a heat exchanger (8). The condensate is then subjected to phase separation (9). The organic phase is discharged as product (11), and the aqueous phase (12) can be used further. The regenerated carrier gas stream (10) is recirculated.

Illustration 1b shows another possible embodiment of the method according to the invention, whereby in this case the gas stripping process takes place in an external gas stripping column (13) connected to the bioreactor. Fermentation solution is thereby fed to the external gas stripping column, and the stripped solution is then recirculated into the bioreactor. All other method steps are analogous to illustration 1a.

In a particularly preferred embodiment in accordance with the method of the invention, the same active material is used as carrier and catalyst for the adsorption and the catalytic reaction. This enables the following further, particularly preferred embodiments of the method according to the invention:

Illustration 2 shows another possible embodiment of the method according to the invention, i.e. the revolver solution in which four (A to D) or more columns are employed. At first, the columns A and B undergo adsorption (1), wherein these columns can be connected in series as well as in parallel. Column C undergoes desorption (2) in that a carrier gas stream is blown in at increased temperatures or reduced pressure. The catalytic reaction takes place in column D, with the desorbed gas stream being blown in. At the end of the cycle time, column B passes on to desorption (2), C to catalytic reaction (3), and D to adsorption (1). Columns D and A are then set for adsorption. After as many cycle times as there are columns, the same column is desorbed again as at the beginning, such that one cycle is complete and a new cycle begins.

Illustration 3 shows another possible embodiment of the method according to the invention in which three (A to C) or more columns are employed and in which desorption and catalytic reaction take place simultaneously in the same column. At first, the columns A and B undergo adsorption (1), wherein these columns can be connected in series as well as in parallel. In column C, the volatile organic compounds are desorbed and at the same time catalytically reacted via temperature increase (3). So that it is possible to set a specific residence time distribution, a portion of the desorbate gas stream is recirculated into column C. At the end of the cycle time, column B passes on to desorption and catalytic reaction (2), and C to adsorption (1). Columns C and A are then set for adsorption. After as many cycle times as there are columns, adsorption again takes place in the same column as at the beginning, such that one cycle is complete and a new cycle begins.

Illustration 4 shows another possible embodiment of the method according to the invention using a two-zone radial adsorber. Adsorption from the gas stream (1) containing the volatile organic compounds takes place in zone A, and desorption and the catalytic reaction forming the product gas stream (2) take place simultaneously in zone B. Rotation of the apparatus causes continuously loaded adsorption material to arrive from the adsorption zone (A) at the desorption and catalytic reaction zone (B), and vice versa.

Illustration 5 shows another possible embodiment of the method according to the invention using an entrained flow reactor having an adsorption zone (A) and a reaction zone (B). Adsorption of the volatile organic compounds from the gas stream (1) takes place in the adsorption zone (A), and desorption and catalytic reaction take place in zone B by blowing in a hot carrier gas stream (2) which entrains the particles, conveying them upwards within the so-called riser. Here, gas (dotted line) and particles (continuous line) are conveyed co-currently. Particle separation takes place at the riser head. The particles then travel back into the adsorption zone (A), such that a closed-loop particle circulation results as a whole.

Illustration 6 shows yet another possible embodiment of the method according to the invention using a moving bed reactor having an adsorption zone (A) and a reaction zone (B). Adsorption of the volatile organic compounds from the carrier gas stream (1) takes place in the cooler adsorption zone (A). The loaded particles then migrate into the warmer reaction zone (B) in which desorption and catalytic reaction take place. The organic products are channelled out of the reactor by a carrier gas stream (2). The particles are conveyed out of the reactor downstream of the reaction zone and conveyed back into the adsorption zone (A) by means of suitable solids conveying techniques, such that a closed-loop particle circulation results as a whole.

In another preferred embodiment, the method according to the invention is furthermore characterized in that one, preferably two, more preferably three, even more preferably four, and again more preferably five or more of the individual method steps are carried out under the following conditions:
a. the fermentation occurs at temperatures between 10 and 70° C., preferably between 20 and 60° C., especially preferably between 30 and 50° C.,
b. the specific aeration rate during gas stripping lies between 0.1 and 10 vvm, preferably between 0.5 and 5 vvm,
c. the temperature during adsorption lies between 10 and 100° C., preferably between 20 and 70° C., and the pressure lies between 0.5 and 10 bar, preferably between 1 and 2 bar,
d. the desorption occurs via temperature increase and/or pressure reduction,
e. the catalytic reaction occurs at a temperature of 150 to 500° C., preferably between 250 and 350° C., at an absolute pressure of 0.5 to 100 bar, preferably between 1 and 5 bar, and a GHSV of 100 to 20000 h⁻¹, preferably between 2000 and 8000 h⁻¹,
f. the condensation takes place via temperature reduction and/or pressure increase,
g. during decantation the organic compounds are separated as the lighter phase.

It is possible in accordance with the invention to combine the condition(s) specified in the previous section with the use of one of the preferred reactors shown in illustrations 1 to 6.

### BRIEF DESCRIPTION OF THE DRAWINGS

Illustration 1 (a and b) shows example embodiments of the method according to the invention with gas stripping in the bioreactor (1a) and with gas stripping in an external gas stripping column (1b).

Illustration 2 shows an embodiment according to the invention having a revolver configuration.

Illustration 3 shows an embodiment according to the invention in which the desorbate gas stream is recirculated into the same column.

Illustration 4 shows an embodiment according to the invention comprising a radial adsorber.

Illustration 5 shows an embodiment according to the invention comprising an entrained flow reactor.

Illustration 6 shows an embodiment according to the invention comprising a moving bed reactor.

Illustration 7 shows how the proportion of ethanol and water is adjusted by means of different adsorption temperatures according to example 1.

Illustration 8 shows the comparison between two fermentation processes using Pachysolen tannophilus without (top) and with continuous ethanol separation via gas stripping and adsorption according to example 2 (bottom; the finely-dashed ethanol sum curve takes into account the sum of ethanol in the solution and bound to the adsorbent).

Illustration 9 shows the influence of the proportion of ethanol in the gaseous desorbate stream on the yield of liquid organic phase, based on the amount of ethanol used according to example 3.

### EXAMPLES

Example 1

### Gas Stripping and Adsorption at Different Temperatures

500 mL of a 5% (w/v) ethanol-water solution was stripped for 24 hours at a volumetric flow rate of 1 L/min. A diaphragm pump (KNF, Germany), a volume flow controller (Swagelok, Germany), and a gas washing bottle (VWR, Germany) were used. The gas stream was passed through a glass column (VWR, Germany) packed with 200 g zeolite granules (ZSM-5, hydrogen form; SiO₂/Al₂O₃=200; binder: bentonite; diameter: 2-4 mm; manufacturer: Süd-Chemie AG, Germany). The gas stream was recirculated into the gas washing bottle in a closed-loop circulation, and therefore the system was closed. The glass column was heated to different temperatures via a heating sleeve (Mohr & Co. GmbH, Germany). Gas stripping in the gas washing bottle took place at 30° C. At the end of the experiment, the ethanol concentration in the solution was determined by gas chromatography (Trace GC, ThermoFischer, Germany). Moreover, the increase in weight was determined for the zeolite and the solution. A mass-balance was then used to calculate the water and ethanol loads of the zeolite and, based thereon, the proportion of water and the proportion of the volatile organic compound ethanol.

Illustration 7 shows the proportions of water obtained, as well as the proportions of volatile organic compounds, as a function of the adsorption temperature. In accordance therewith, the proportion of water and the proportion of volatile organic compounds can be set via the adsorption temperature.

### Example 2

### In Situ Fermentation with Gas Stripping and Adsorption

Pachysolen tannophilus (DSM 70352, DSMZ, Brunswick, Germany) was fermented for 100 hours at 30° C. and pH 6 with and without the continuous separation of ethanol via gas stripping and adsorption under otherwise identical conditions. Pretreated and hydrolised lignocellulosic biomass containing approx. 70 g/L glucose and approx. 30 g/L xylose was employed as substrate. Bioreactors having a filling volume of 0.8 L each were used as bioreactors. In the case of fermentation with continuous separation, gas stripping was carried out at a specific aeration rate of 1 vvm using a diaphragm pump (KNF, Germany). Just as in example 1, the gas stream was passed through a glass column and then recirculated. The glass column was packed with 535 g zeolite granules (ZSM-5, hydrogen form; SiO₂/Al₂O₃=200; binder: bentonite; diameter: 2-4 mm; manufacturer: Süd-Chemie AG, Germany). Samples were taken during fermentation and the ethanol content was quantified by gas chromatography and the sugars by HPLC. In addition, the increase in weight of the zeolite and the proportion of water of the adsorbed mixture were determined by Karl Fischer titration (Schott Instruments, Germany). It is known from preliminary experiments that only water and ethanol are adsorbed under the given conditions. As a result, the proportion of ethanol can be concluded from the water content.

Illustration 8 shows the concentration curves obtained. It can be seen therefrom that carrying out fermentation, gas stripping and adsorption at the same time is advantageous and that under the given conditions higher space-time yields are achieved by fermentation with continuous separation of the volatile compounds.

### Example 3

### Catalytic Reaction

A packed bed reactor (length=50 cm, inner diameter=2.5 cm) from the firm ILS—Integrated Lab Solutions GmbH was used for catalytic reaction. By means of an HPLC pump (Smartline Pump 100, Wissenschaftliche Gerätebau Dr. Ing. Herbert Knauer GmbH), the liquid model desorbate (40% by weight of EtOH, 60% by weight of water) was added portionwise to the reaction tube where it was evaporated by means of a heated inert SiC prepacking, mixed with nitrogen such that 4% by weight of nitrogen were present, and brought to the reaction temperature of 300° C. and the absolute pressure of 3 bar. The gaseous desorbate stream thus obtained was ultimately directed over a packing of 10 g zeolite extrudate (zeolite ZSM-5, hydrogen form, SiO₂/Al₂O₃=90; binder Al₂O₃; diameter= 1/16 inch; manufacturer: Süd-Chemie AG) at a gas hourly space velocity (GHSV) of 5800 h⁻¹. The gaseous product stream was cooled to 10° C. in a gas-liquid separator downstream of the packed bed reactor, thereby condensing the liquid products and separating them from the gaseous products. The liquid organic phase was then separated from the aqueous phase by decantation. The experiment was carried out over a total time-on-stream (TOS) of 24 h.

The liquid organic phase accumulated during this length of time was in the end analyzed by gas chromatography coupled to mass spectrometry (see table 1 for composition). As the evaluation has shown, an ethanol conversion of >99% and a yield of liquid organic phase of 34% by weight, based on the amount of ethanol used, were achieved under these experimental conditions.

TABLE 1
Composition of the liquid organic phase

| Substance Class | Proportion [GC Area %] |
|---|---|
| Unbranched alkanes (<C5) | 1.6 |
| Unbranched alkanes (C5-C10) | 3.8 |
| Branched alkanes (C5-C10) | 30.3 |
| Branched olefins (C5-C10) | 2.7 |
| Cyclic hydrocarbons | 2.9 |
| Benzene | 0.3 |
| Toluene | 3.8 |
| Xylenes | 10.8 |
| Monoalkylated aromatics | 43.8 |
| (without toluene, xylenes) | |

In a further experiment, the proportion of ethanol in the gaseous desorbate stream was varied under otherwise identical conditions by evaporating different model desorbates in the heated prepacking and mixing them with different amounts of nitrogen. Illustration 9 shows the influence of the proportion of ethanol in the gaseous desorbate stream on the yield of liquid organic phase, based on the amount of ethanol used. It can be seen that a higher proportion of ethanol has a beneficial effect on the yield of liquid organic phase.

### Example 4

### Adsorption to Zeolite

500 mL of a 5% (w/v) ethanol-water solution was stripped with the set-up explained in example 1 for 24 hours at 30° C. and at 1 vvm using a diaphragm pump (KNF Neuberger, Freiburg, Germany) and a volume flow controller (Swagelok, Garching, Germany). In this process, the gas stream was passed through a glass column (Gassner Glastechnik, Munich, Germany) which was filled in each case with 200 g adsorbent (zeolite with SiO₂/Al₂O₃). The column was brought to a temperature of 40° C. by means of a heating sleeve (Mohr & Co. GmbH, Germany). After 24 hours the experiment was terminated, the increase in weight of the packing determined and the ethanol concentration quantified by gas chromatography (Trace GC, Thermo Fisher). Since the system is closed, the ethanol stripped from the solution must have been adsorbed on the adsorbent. The remaining increase in weight is due to water. The adsorbed amounts of ethanol and water were thus calculated by mass balance and the following capacities determined.

| | Capacity for | Capacity for |
|---|---|---|
| | EtOH [%] | Water [%] |
| Zeolite with SiO₂/Al₂O₃ = 1000 | 6.83 | 0.87 |

As can be seen, ethanol is selectively adsorbed compared to water. The zeolite is thus particularly well suited as an adsorbent.

### Example 5

40 g of a zeolite according to the invention having a SiO₂/Al₂O₃ ratio of 1000 is added in 400 mL of a 5% (w/v) aqueous ammonia solution. The mixture is suspended at room temperature for one hour. Following this, the zeolite is separated again. 50 mL of the remaining solution is in each case titrated four times with 5 molar hydrochloric acid, which is added by means of a burette, and methyl red as a pH indicator. Upon the change in indicator colour which marks the equivalence point, the volume of hydrochloric acid added is read. The amount of hydrochloric acid added which corresponds to the amount of ammonia is calculated based thereon; this is used in turn to determine the concentration of the ammonia solution.

An ammonia concentration of 46.15+/−0.88 g/L is obtained.

Four-fold titration is repeated for the ammonia solution used in this experiment which was not contacted with the zeolite. An ammonia concentration of 46.13+/−0.33 g/L results here.

The comparison shows that the zeolite did not adsorb any ammonia since otherwise the concentration of ammonia in the solution contacted with the zeolite would have had to be lower.

Examples 4 and 5 together show that the zeolite is suited for selective adsorption of ethanol and that at the same time adsorption of the undesired compound ammonia to this adsorbent is negligible.

1. A method for producing organic compounds, comprising the following method steps:
a. fermentative conversion of biomass to volatile organic compounds in a bioreactor;
b. removal of the volatile organic compounds by gas stripping using a carrier gas;
c. adsorption of the volatile organic compounds from the gas stream;
d. desorption of the adsorbed volatile organic compounds from the adsorber;
e. catalytic reaction of the volatile organic compounds.

2. The method according to claim 1, wherein in method step d the proportion of volatile organic compounds in the desorbate stream lies between 10% (w/w) and 90% (w/w).

3. The method according to claim 2, wherein following method steps a to e, condensation of the product stream takes place, and wherein following condensation phase separation takes place.

4. The method according to claim 1, wherein method steps a to e proceed in parallel.

5. The method according to claim 1, wherein the volatile organic compounds are alcohols and/or ketones and/or aldehydes and/or organic acids.

6. The method according to claim 1, wherein the carrier gas(es) is/are recirculated following the adsorption and/or following the catalytic reaction and/or the fermentation exhaust gases are used as carrier gas.

7. The method according to claim 3, further characterized in that at least one of the individual method steps is carried out under the following conditions:
a. the fermentation occurs at temperatures between 10 and 70° C., preferably between 20 and 60° C., especially preferably between 30 and 50° C.,
b. the specific gassing rate during gas stripping lies between 0.1 and 10 vvm, preferably between 0.5 and 5 vvm,
c. the temperature during adsorption lies between 10 and 100° C., preferably between 20 and 70° C., and the pressure lies between 0.5 and 10 bar, preferably between 1 and 2 bar,
d. the desorption occurs via temperature increase and/or pressure reduction,
e. the catalytic reaction occurs at a temperature of 150 to 500° C., preferably between 250 and 350° C., at an absolute pressure of 0.5 to 100 bar, preferably between 1 and 5 bar, and a GHSV of 100 to 20000 h-1, preferably between 2000 and 8000 h-1,
f. the condensation takes place via temperature reduction and/or pressure increase,
g. during decantation the organic compounds are separated as the lighter phase.

8. The method according to claim 1, wherein the adsorber is a zeolite.

9. The method according to claim 1, wherein the catalyst is a zeolite.

10. The method according to claim 8, wherein the adsorber is selected so that there is no adsorption of ammonia for the catalytic reaction.

11. The method according to claim 1, wherein the zeolite adsorber and the zeolite catalyst are of the same material.

12. The method according to claim 11, wherein the zeolite material is filled into several parallel columns which alternate, as in a revolver configuration, at staggered time intervals between several method steps, these method steps being selected from adsorption, desorption, catalytic reaction and possibly regeneration.

13. The method according to claim 11, wherein adsorption, desorption and catalytic reaction each take place at staggered time intervals in the same column.

14. The method according to claim 11, wherein the adsorption, desorption and catalytic reaction take place in a single apparatus.

15. The method according to claim 14, wherein the apparatus is a radial adsorber, moving bed reactor or an entrained flow reactor.

16. The method according to claim 9, wherein the catalyst is an MFI-type zeolite.

17. The method according to claim 16, wherein the catalyst is an MFI-type zeolite in the hydrogen form.

18. The method according to claim 9, wherein the zeolite catalyst has a SiO₂/Al₂O₃ ratio of 5 to 1000

19. The method according to claim 18, wherein the zeolite catalyst has a SiO₂/Al₂O₃ ratio of 20 to 200.

20. The method according to claim 2, wherein following method steps a to e, condensation of the product stream takes place.
